SEQUENCE LISTING (2 sequences)

SEQ ID NO: 1 - DNA, 29 bp, Artificial Sequence
  5′ primer for amplififcation and subcloning of extracellular domain of human CD36.
aagaattccgagacctgcttatccagaag

SEQ ID NO: 2 - DNA, 35 bp, Artificial Sequence
  3′ primer for amplififcation and subcloning of extracellular domain of human CD36.
agcggccgcttagtttatttttccagttacttgac

(19) United States
(12) Patent Application Publication
(10) Pub. No.: US 2017/0363646 A1
(43) Pub. Date: Dec. 21, 2017

(54) METHOD FOR DETECTING MOLECULES THAT INHIBIT THE INTERACTION OF CD36 PROTEIN WITH AMYLOID BETA

(71) Applicant: Instituto de Investigaciones Cientificas y Servicios de Alto Technologia AIP (Indicasat AIP), Clayton, PA (US)

(72) Inventors: Patricia LLANES FERNANDEZ, Clayton (PA); Ricardo LLEONART CRUZ, Clayton (PA); Deborah Doens, Clayton (PA)

(21) Appl. No.: 15/650,072

(22) Filed: Jul. 14, 2017

Related U.S. Application Data
(63) Continuation of application No. PCT/PA2016/000001, filed on Jan. 14, 2016.

(30) Foreign Application Priority Data
Jan. 14, 2015 (PA) 90493-1

(51) Int. Cl. G01N 33/68

(52) U.S. Cl. CPC G01N 33/6896; G01N 2500/20; G01N 2333/4709; G01N 2333/70596; G01N 2500/02

(57) ABSTRACT

Disclosed is a method for identifying inhibitors of the interaction of CD36 receptor and amyloid-beta protein (Aβ), which consists in: immobilizing CD36 on a polystyrene plate; and detecting by colorimetric, the interaction thereof with fibrillar Aβ (fAβ), using a polyclonal anti-Aβ antibody and a secondary antibody conjugated to horseradish peroxidase (HRP). The inhibitors identified are potential agents for the treatment of Alzheimer's disease.

### CROSS REFERENCE TO RELATED APPLICATIONS

This application is a continuation of and claims the benefit of priority to PCT/PA2016/000001, filed 14 Jan. 2016, which claims priority to PA 90493-01, filed 14 Jan. 2015, the disclosures of which are incorporated by reference as if fully set forth herein in their entirety.

### FIELD OF THE INVENTION

The present invention is related to the area of Neuroimmunology particularly in the development of a procedure for identifying molecules that interfere with the interaction between the CD36 receptor and the amyloid beta.

### BACKGROUND OF THE INVENTION

Alzheimer is a neurodegenerative disease, which accounts for the most common type of dementia in the elderly. It has been estimated that more than 35 million people in the world suffer from Alzheimer's disease (1). It is estimated that approximately 95% of Alzheimer's cases are sporadic, which means, there is no genetic cause established. The toxic agent that triggers the disease remains unknown, although it has been proven that the presence of amyloid beta (Aβ) plaques constitute one of its typical pathologic features. These plaques are formed by fibrils and the relevance of these structures has been demonstrated in the pathogenesis of Alzheimer's disease (2).

The presence of inflammation associated with glial cells (mainly microglia), which are in close relationship with Aβ plaques (3) has been demonstrated as well. The presence of activated microglial cells surrounding deposits of Aβ has been observed in the brain of Alzheimer's patients as well as in animal models of the disease (4). An increase in the concentration of inflammatory mediators such as prostaglandins, pentraxins, complement components, cytokines, chemokines, and proteases among others, has been reported in affected areas of the brain in patients with Alzheimer's disease (5).

The recognition of fibrillar Aβ by microglia is an essential step of the inflammatory process that contributes to the pathogenesis of the disease. The activation of the cells of the innate immune system, as in the case of microglia, occurs mainly through detection of host or pathogen-derived molecules through receptors expressed by these cells.

The role of the CD36 receptor in the activation of the microglia induced by fibrillar Aβ has been described. The CD36 receptor is expressed in human fetal microglia and in the brain of patients with Alzheimer's disease (6). Cells that express CD36 are able to interact with surfaces covered with Aβ fibrils. The genetic deficiency of CD36 provokes a reduction in the production of inflammatory mediators by microglia and peritoneal macrophages stimulated in vitro by Aβ fibrils, and in the recruitment of these cells in vivo after the Aβ experimental injection (7).

The interaction between Aβ and CD36 triggers a cascade of intracellular signaling events that implicate members of the family of protein kinase Src and MAPKs (mitogen-activated protein kinase). The disruption of this interaction leads to inhibit the production of chemokines and oxygen reactive species by in vitro stimulated macrophages and the recruitment of microglia to sites of Aβ accumulation in vivo (8).

The CD36 receptor has been involved in the interaction with other molecules, including low-density lipoproteins, thrombospondin, long-chain fatty acids, growth hormone secretagogues (GSHs) peptides, high-density lipoproteins, among others (9).

The interaction of synthetic peptides derived from GSHs with CD36 interferes with the production of interleukin (IL)-1β and IL-6 induced by Aβ in N9 microglial cell line (10). Therefore, pharmacological inhibition of the interaction between CD36 and Aβ could have a positive impact in the treatment and/or prevention of Alzheimer's disease.

Immunotherapies described up to this moment, are based on the administration of anti-Aβ antibodies capable of reducing the burden of Aβ in the plaques (5). So far, no conclusive results have been obtained from these studies, and the mechanism by which these antibodies exert their effect is still unclear. Amongst the anti-inflammatory therapies tested, none has been proposed with the purpose to find compounds capable to inhibit the Aβ-CD36 interaction, in spite of its significance triggering the inflammation associated with Alzheimer's disease.

Currently, approved drugs for the treatment of Alzheimer's disease have a very short efficiency period and are only effective on about 50% of AD patients. Many clinical trials conducted for testing of several drugs have not provided conclusive results. This invention presents the development of an assay intended for the rapid identification of molecules capable of inhibiting the interaction between Aβ and CD36, as a first step in the discovery of new molecules with therapeutic potential for Alzheimer.

The patent application No 20040115737 at the USPTO, entitled “Cd36 as a heat shock protein receptor and uses thereof”, with the authorship of Panjwani Naveed, and dated on Jun. 17, 2004, describes an assay based on cell lines to identify compounds that interact with CD36 and modulate their interaction with the heat shock protein (HSP), as well as the possible use of these compounds for the treatment of immune and proliferative disorders. Different from that patent application, our solution is focused on an in vitro assay capable of identifying molecules able to inhibit the interaction between CD36 and the amyloid beta, which would enable the development of new drugs against Alzheimer's disease.

The U.S. Pat. No. 8,124,358, entitled “Methods of screening for compounds that inhibit binding between amyloid-beta (Abeta) and FC-gamma receptor IIB (Fc gamma RIIb)” with the authorship of Yong-Keun Jung and Sungmin Song, and dated on Feb. 28, 2012, describes an assay based on cell lines aimed to identify compounds that inhibit the binding between amyloid beta and the Fc gamma receptor IIb, with the objective to improve the diagnosis, prevention and treatment of Alzheimer's disease. In contrast to that patent, this proposal is based on the interaction of the amyloid beta with another receptor, the CD36, and the proposed assay is conducted entirely in vitro, which makes it much faster and convenient for the identification of new inhibitory molecules. The possibility of conducting an in vitro assay allows studying a higher number of compounds in less time than when the assay is based on cell lines.

### BRIEF SUMMARY OF THE INVENTION

The main objective of the invention's patent application is the endowment of a procedure and/or method that allows the identification of molecules capable of interrupting the interaction between the CD36 receptor and the amyloid beta.

### Advantages of the Invention

The invention's patent application for “Procedure for detecting molecules that inhibit the interaction between the CD36 protein and the amyloid beta” presents the following advantages in relation to the State of the Art:
Large-scale identification of molecules able of interrupting the interaction between CD36 receptor and amyloid beta over a short period of time.
It allows detecting the interaction of CD36 with soluble amyloid beta fibrils (fAβ) through the use of a polyclonal antibody, which recognizes the Aβ and a secondary antibody conjugated to the enzyme horseradish peroxidase (HRP), thus detecting the rCD36-fAβ complex.
Rapid identification of potential agents for the treatment of Alzheimer's disease.

### BRIEF DESCRIPTION OF THE DRAWINGS

FIG. 1. Schematic representation of the assay. 1. (Substrate), 2. (color development), 3 (an antibody specific for amyloid beta, and an anti-antibody labeled with HRP or an antibody specific for amyloid beta conjugated to HRP), 4. (amyloid beta), 5 (CD36).

FIG. 2. Graph depicting the optimization result of CD36 and Aβ concentrations in the assay. Each letter corresponds to a series of absorbance values in serial dilutions 1:2 of rCD36 and Aβ as described in Example 2.

FIG. 3. Graph depicting the result of the DMSO effect on the assay. Each letter corresponds to a series of absorbance values in serial dilutions 1:2 of rCD36 and Aβ as described in examples 2 and 3.

FIG. 4. Tolerance of the assay at increasing concentrations of DMSO.

FIG. 5. Graph depicting the optimization results of the concentrations of antibodies in the assay. Each letter corresponds to a series of absorbance values in serial dilutions 1:2 of primary and secondary antibodies as described in Example 4.

FIG. 6. Optimization of the ursolic acid (A.U.) incubation conditions.

FIG. 7. Optimization of the incubation time of the ursolic acid (A.U.).

FIG. 8. Optimization of concentrations of CD36 and amyloid beta using OPD as a substrate developer.

### DETAILED DESCRIPTION OF THE INVENTION

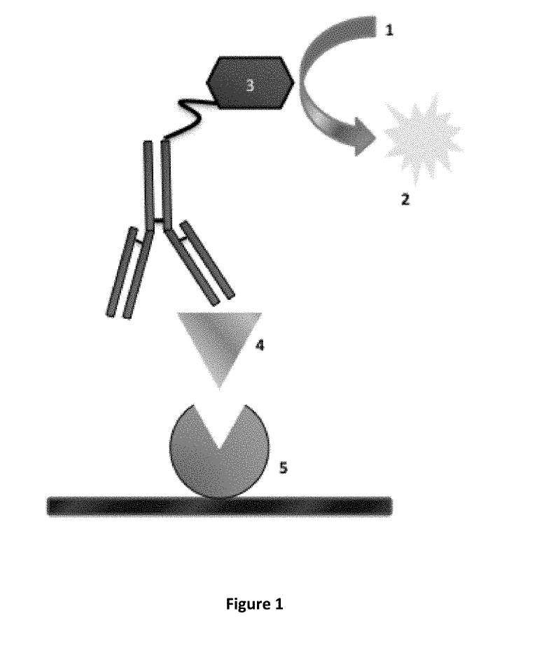

The invention consists in the development of a rapid colorimetric procedure that through the adhesion of the recombinant protein CD36 to a polystyrene plate enables the evaluation of molecules that can inhibit the interaction of such protein and the amyloid beta. The process or method of this invention is based on the interaction of A) recombinant extracellular portion of the CD36 protein with B) the Aβ peptide in its fibril state. The complex is revealed using a primary polyclonal antibody and a secondary antibody conjugated to the peroxidase enzyme that specifically recognizes the primary antibody. The presence of compounds that inhibit the ligand-receptor interaction in the reaction produces a reduction of the colorimetric signal generated after the addition of the substrate (FIG. 1). The fibrillar Aβ is prepared on the basis of the synthetic peptide incubated at 37° C., in distilled water. The fibrils are prepared by controlling the polymerization of the monomers, which is monitored through the Thioflavin T fluorescent assay (11).

The rapid and high-throughput procedure for the detection of inhibitory molecules of the CD36-amyloid beta interaction, develops through the following steps:
the immobilization of one of the components (CD36 or Aβ) on a solid surface
the addition of the other component of the interaction in soluble phase in presence or not, of potential inhibitors and
the detection of the complex through a marker molecule.

The solid surface can be made of glass, polystyrene, modified polystyrene, or any other type of surface suitable for immobilizing the components of the interaction and their subsequent detection, by containing a variable amount (96, 384 or 1536) of wells.

The Aβ species may be 1-40 or 1-42 and can be found in any of its polymerization states, in the form of monomer, oligomer or fibril.

The use of a complex formed by an antibody conjugated to an enzyme that generates a detectable product, which can be colored or of any other type. The marker molecule consists of a primary antibody specific for the soluble component of the interaction (CD36 or Aβ) and a secondary antibody, specific for the primary antibody, conjugated to an enzyme that can be peroxidase, alkaline phosphatase or another enzyme suitable for its detection.

The following examples are described within the framework of this invention:

### Example 1

For the cloning of the gene that encodes for the extracellular domain of the CD36 protein, corresponding to Gly30-Asn439, the total RNA was extracted from the THP-1 cells following the procedure previously described (12). This RNA was used as a template to synthesize complementary DNA (cDNA) using established methods (13). Then, the fragment corresponding to the extracellular domain of the human CD36 protein was amplified through a polymerase chain reaction (PCR) using as primers the oligonucleotides (a) AAGAATTCCGAGACCTGCTTATCCAGAAG 3′ and (b) 5′ AGCGGCCGCTTAGTTTATTTTTCCAGTTACTTGAC 3′ (14). The primer that anneals at the 5′ end of the gene contains a recognition site for the restriction enzyme EcoRI, and the primer that hybridizes with the 3′ end of the gene contains a site for the restriction enzyme NotI, in order to facilitate the subsequent cloning of the fragment. The PCR product was cloned into the vector pGEM-T, generating the vector pGEM-T-CD36. The vectors pGEM-T-CD36 and pET30a(+) were digested with the restriction enzymes EcoRI and NotI, and the corresponding fragments were ligated by T4 DNA ligase in order to obtain the pET30-CD36 vector. The resulting expression plasmid expresses the extracellular portion of the CD36 protein fused in its 3′ end to a tail of six residues of the amino acid histidine, which allows the purification and detection of the CD36 protein. The pET30-CD36 expression vector was transformed into competent cells of the strain of Escherichia coli (E. coli) BL21 (DE3) and the expression of the protein was induced with IPTG (Isopropyl β-D-1-thiogalactopyranoside). The purification of the CD36 fragment was performed by immobilized-metal affinity chromatography (IMAC) as it has been previously described (15). After the purification of the recombinant protein CD36 (rCD36) under denaturing conditions, its renaturation was completed by dilution in a phosphate buffer solution (PBS).

### Example 2

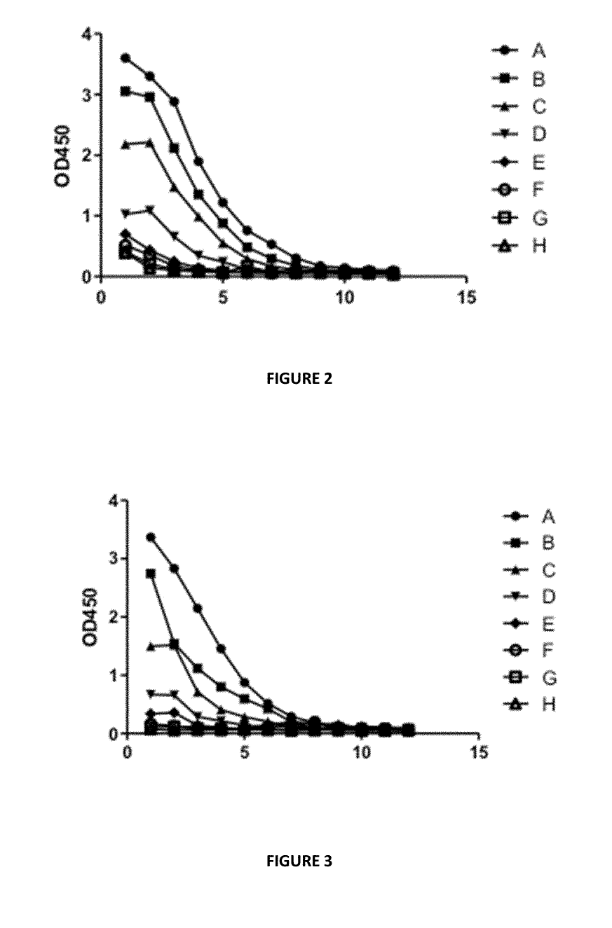

In order to start the optimization of the assay, the optimum concentrations of rCD36 and amyloid beta fibrils (fAβ) were established. The first step was the immobilization of rCD36 diluted in PBS buffer to a 96-well polystyrene plate (MaxiSorp). Serial dilutions (1:2) of the recombinant protein were added in the vertical direction of the plate starting at 30 μg/ml and up to 0.4 μg/ml, which was incubated for 16h-18h at a temperature of 4° C. Subsequently, we proceeded to block the plate with 3% skim milk in PBS buffer. Then added fAβ1-42, prepared in accordance with the procedures established in the literature (16), in serial dilutions (1:2) in the horizontal direction of the plate starting at 2.5 μM and up to 2.5 nM. This procedure of crossed dilutions (chessboard titration) allowed us to identify the optimal concentrations of rCD36 and fAβ1-42. For this assay, fixed concentrations of the primary (2 μg/ml) and secondary (1:1000) antibodies, obtained commercially, were maintained. Optimal concentrations were considered to be the ones with significant color intensity in the absence of inhibition (approximately A450˜1 in 15 minutes) (FIG. 2). In these conditions the optimal concentrations of rCD36 and fAβ1-42 were 15 μg/ml and 0.2 μM respectively.

### Example 3

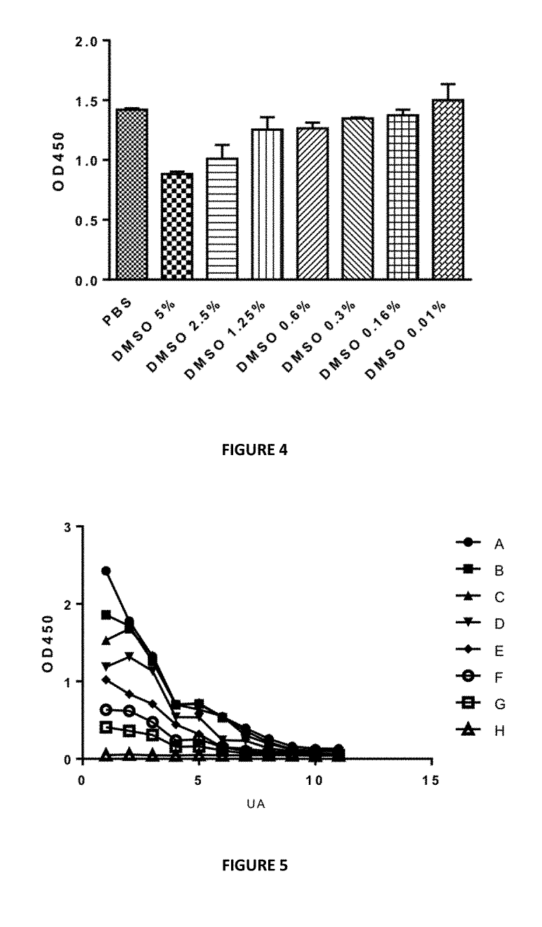

Considering that the vast majority of the natural or synthetic compounds that will be tested in the screening phase are diluted in Dimethyl Sulfoxide (DMSO), the tolerance of the assay to this solvent was evaluated. First, the assay sensitivity was evaluated at a concentration of 0.1% DMSO in conjugated serial dilutions where concentrations of rCD36 and fAβ1-42 varied, similar to the description given in example 2 (FIG. 3). The DMSO was added after the blocking step, 30 minutes prior to adding fAβ1-42, and maintained during the incubation period of the latter. In these conditions it was observed that the DMSO slightly decreased the sensitivity of the assay since the optimum concentration of fAβ1-42 increased to 0.5 μM. Nonetheless, the optimum concentration of rCD36 remained at 15 μg/ml. Later, the tolerance of the assay was evaluated at different doses of DMSO. In order to accomplish this, serial dilutions (1:2) of DMSO from 5% up to 0.01% were tested. In this case, fixed concentrations of rCD36 and fAβ1-42 (15 μg/ml and 0.5 μM respectively) were used, which correspond to the concentrations previously determined. We observed that our assay is sensitive to DMSO concentrations greater than 1.25% (FIG. 4). From this moment on, concentrations of rCD36 and fAβ142 were established at 15 μg/ml and 0.5 μM respectively, and for the rest of the optimizations the fAβ1-42 was incubated in the presence of 0.7% DMSO.

### Example 4

Once optimal concentrations of rCD36 and fAβ1-42 were identified, we proceeded with the optimization of the concentrations of the anti-Aβ rabbit polyclonal primary antibody (GenScript) and for the anti-rabbit secondary antibody conjugated to peroxidase (Santa Cruz Biotechnology). In order to accomplish this, we used the same procedure of conjugated dilutions as illustrated in FIG. 3. Serial dilutions (1:2) of the anti-Aβ primary antibody were added in the horizontal direction of the plate starting at 2 μg/ml and up to 2 ng/ml. The anti-rabbit secondary antibody was added in the vertical direction of the plate in serial dilutions (1:2) from 1:1000 up to 1:64000 (FIG. 5). Thus, selecting the concentration of 0.3 μg/ml for the anti-Aβ polyclonal primary antibody and the 1/4000 dilution for the anti-rabbit secondary antibody.

### Example 5

Continuing with the optimization process of the assay, different buffers were evaluated to block the plate. Blocking is an important step of the assay as it enables to decrease the nonspecific signal (noise or background) caused by nonspecific bindings to the polystyrene plate. This step consists of the incubation of the plate in which the rCD36 has been previously immobilized with a buffer that contains high concentrations of proteins that will adhere to surfaces free of rCD36. The use of detergents in this step has also proven efficiency in decreasing the noise. Three variants were tested for the optimization of the blocking buffer (i) PBS-Skim Milk 3%; (ii) PBS-Tween 20 (0.05%) and 3% skim milk and (iii) PBS-Tween 20 (0.05%). For this optimization we used a fixed concentration of rCD36 and serial dilutions of fAβ1-42, added in duplicate. Although the behavior of the curve obtained after the blocking with the buffer (iii) was the expected, we decided to use the blocking buffer (i) for the rest of the experiments, as it shows lowest variability.

### Example 6

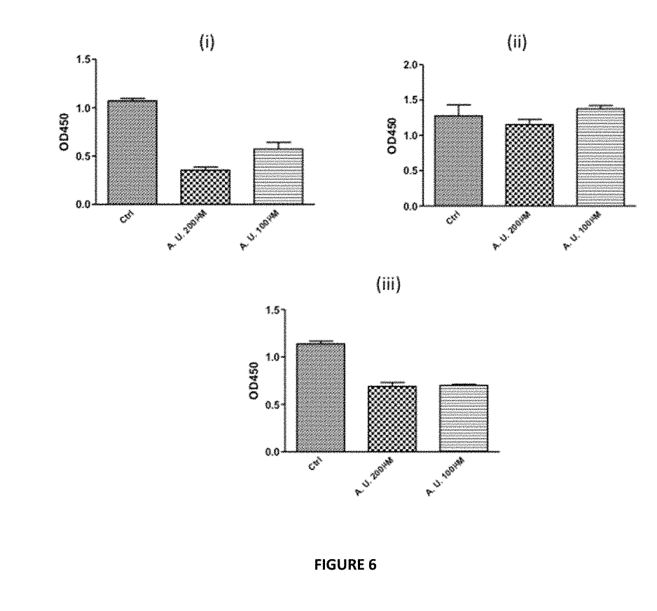

It has been previously demonstrated that the ursolic acid is an inhibitor of the interaction between CD36 and fAβ1-42 (17). Through a cell-based assay, these authors demonstrated that this compound inhibits such interaction, and that this inhibition impacts on the Aβ-induced nitric oxide production in a microglial cell line. To assess the effectiveness of our assay the ursolic acid was used as an inhibition control of the interaction of rCD36-fAβ1-42. First, we evaluated the most appropriate time-of-addition of the ursolic acid on the plate. Three different conditions were tested: (i) the ursolic acid was added after the blocking step, it was incubated for 30 minutes prior to the addition of fAβ1-42 and it was maintained throughout the complete incubation time; (ii) the ursolic acid was added after the blocking step, it was incubated for 30 minutes and it was removed before incubation with fAβ1-42, and (iii) the ursolic acid and fAβ1-42 were adding at the same time after the blocking step and were maintained throughout incubation time (FIG. 6). Two concentrations of ursolic acid (100 and 200 μM) were tested for each condition. It was observed that the optimal incubation condition of the inhibitor corresponds to option (i). This was the condition established for the incubation of molecules during the screening.

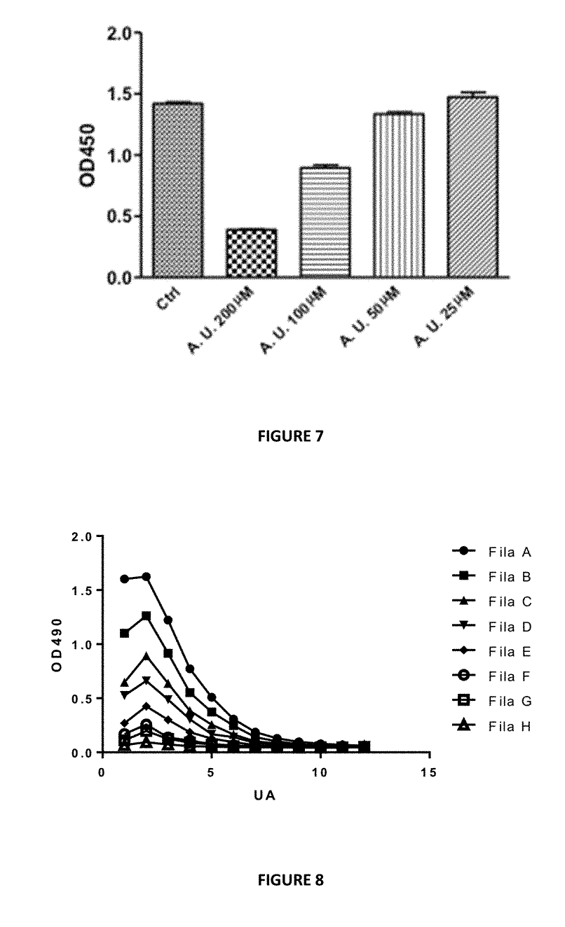

Afterwards, ursolic acid concentrations that would be capable of inhibiting the rCD36-fAβ142 interaction in this assay were evaluated. Increasing concentrations of this inhibitor varying from 25 μM to 200 μM were tested and added to the plate in the same predetermined incubation conditions. We observed a significant decrease (p<0.05 calculated by t-test) of the interaction between rCD36 and fAβ1-42 at ursolic acid concentrations of 100 and 200 μM (FIG. 7). The ursolic acid concentration of 200 μM was defined as the assay positive control.

### Example 7

We tested two substrates of the peroxidase enzyme in order to detect the presence of the secondary antibody, the 3.3′, 5.5′,-Tetramethylbenzidine (TMB), and the o-Phenylenediamine (OPD). We used the same procedure of conjugated dilutions as illustrated in FIG. 3, varying the concentrations of rCD36 and fAβ1-42, as described in Example 2. The absorbance was measured at 490 nm in the case of the OPD, or at 450 nm in the case of the TMB, and the optimal concentrations were considered to be those in which absorbance around 1 was obtained. The sensitivity of the assay varied slightly when the OPD was used as substrate for the peroxidase, since the optimum concentration of fAβ1-42 in these conditions was 0.8 μM. However, the optimum concentration of rCD36 remained at 15 μg/ml (FIG. 8).

### Example 8

After the optimization described previously, the assay developed in this invention allowed us to test a large number of compounds for its capacity to inhibit the CD36-fAβ interaction over a period of time no higher than 24 hours. The rCD36 is immobilized on the polystyrene plate, incubating it during 16-18 hours at a temperature of 4° C. Subsequently, the plate is washed 3 times with PBS-Tween 20 and blocked for 2 hours in the presence of 3% skim milk in PBS buffer. After 3 washings, the compounds to be evaluated are incubated for 30 minutes. Next, the fAβ is added to the wells and incubated for a period of 2 hours followed by 3 washings. Then, the anti-Aβ primary antibody is added and incubated for a period of 2 hours more and washed in the same manner. The secondary antibody conjugated to peroxidase is added and incubated for 2 additional hours. After 3 washings the plate is incubated with the substrate of the enzyme, in this case TMB, for a maximum period of 30 minutes, the reaction stops by adding HCl 1N and the absorbance is read at 450 nm.

### REFERENCES

1. http://www.alz.co.uk/research/statistics.html
2. Hardy J et al. 2002 Science 297, 353-356.
3. Malm T et al. 2010 Brain 58, 889-900.
4. Bennett D A et al. 2006 Neurology 66, 1837-1844.
5. Citron M 2010 Nat. Rev. 9, 387-398.
6. Coraci I S et al. 2002 Am J Pathol 160, 101-112.
7. El Khoury J B et al. 2003 J Exp Med 197, 1657-1666.
8. Moore K J et al. 2002 J Biol Chem 277, 47373-47379.
9. Febbraio M, Silverstein R L 2007 Int J Biochem Cell Biol 39, 2012-2030.
10. Bulgarelli I et al. 2009 J Neurosci Res 87, 2718-2727.
11. Kuznetsova I M et al. 2012 PLoS One 7, e30724.
12. Chomczynski P, Sacchi N 1987 Anal Biochem 162, 156-159.
13. Sambrook J, Fritsch E F, Maniatis T 1989 Molecular cloning: a laboratory manual. Cold Spring Harbor Laboratory, Cold Spring Harbor, N.Y.
14. Wang L et al. 2010 J Biomol Screen 15, 239-250.
15. Roe S. 2001 Protein Purification Techniques, Oxford University Press, Oxford.
16. Jana M et al. 2008 J Immunol 181, 7254-7262.
17. Wilkinson K et al. 2011 J Biol Chem 286, 34914-34922

1. A method for detecting a molecule that inhibits CD36-amyloid beta (Aβ) peptide interactions in an assay, the method comprising:
(i) immobilizing the CD36 on a solid surface of the assay;
(ii) adding the Aβ peptide in its fibril state and a candidate inhibitor molecule to the soluble phase of the assay;
(iii) adding a primary antibody specific for a CD36-Aβ complex, and a secondary antibody labeled with a marker molecule that recognizes the primary antibody;
(iv) adding a substrate that produces a colorimetric signal in the presence of the CD36-Aβ complex; and
(v) detecting the presence of the colorimetric signal,
wherein a reduction or absence of a colorimetric signal is indicative that the candidate molecule is an inhibitor of CD36-Aβ interactions.

2. The method of claim 1, wherein said solid surface is composed of a material selected from the group consisting of glass, polystyrene, and modified polystyrene.

3. The method of claim 1, wherein said CD36 is a recombinant CD36 peptide corresponding to the extracellular portion of CD36.

4. The method of claim 1, wherein said Aβ peptide is selected from Aβ(1-40) or Aβ(1-42).

5. The method of claim 1, wherein said primary antibody is a polyclonal antibody.

6. The method of claim 1, wherein said marker molecule is an enzyme that generates a detectable product that can be colored.

7. The method of claim 6, wherein said enzyme is peroxidase or alkaline phosphatase.

8. A method for detecting a molecule that inhibits CD36-amyloid beta (Aβ) peptide interactions in an assay, the method comprising:
(i) immobilizing the Aβ peptide on a solid surface of the assay;
(ii) adding the CD36 and a candidate inhibitor molecule to the soluble phase of the assay, wherein said CD36 is a recombinant peptide corresponding to the extracellular portion of CD36;
(iii) adding a primary antibody specific for a CD36-Aβ complex, and a secondary antibody labeled with a marker molecule that recognizes the primary antibody;
(iv) adding a substrate that produces a colorimetric signal in the presence of the CD36-Aβ complex; and
(v) detecting the presence of the colorimetric signal,
wherein a reduction or absence of a colorimetric signal is indicative that the candidate molecule is an inhibitor of CD36-Aβ interactions.

9. The method of claim 8, wherein said solid surface is composed of a material selected from the group consisting of glass, polystyrene, and modified polystyrene.

10. The method of claim 8, wherein said Aβ peptide is in a polymerization state selected from the group consisting of: monomer, oligomer, and fibril.

11. The method of claim 10, wherein said Aβ peptide is selected from Aβ(1-40) or Aβ(1-42).

12. The method of claim 8, wherein said primary antibody is a polyclonal antibody.

13. The method of claim 8, wherein said marker molecule is an enzyme that generates a detectable product that can be colored.

14. The method of claim 13, wherein said enzyme is peroxidase, alkaline phosphatase.
